(19) United States
(12) Patent Application Publication
(10) Pub. No.: US 2007/0203389 A1
(43) Pub. Date: Aug. 30, 2007

(54) Medical device

(76) Inventor: Harold Bergman, Vancouver (CA)

Correspondence Address: LONG AND CAMERON, SUITE 1401 - 1166 ALBERNI STREET, VANCOUVER, BC, V6E 3Z3, CA

(21) Appl. No.: 11/363,318

(22) Filed: Feb. 28, 2006

(51) Int. Cl. A61N 1/00; A61B 17/52; A61N 2/00

(52) U.S. Cl. 600/13; 600/9

(57) ABSTRACT

A medical device and a method for therapeutic treatment within an oral cavity employ a control circuit for generating an electromagnetic field , the circuit including an electrical power source and being integrated in a prosthetic appliance shaped to fit onto a patient's dentition.

### BACKGROUND OF THE INVENTION

1. Field of the Invention

The present invention relates to a medical device used intra-orally for therapeutic purposes.

2. Description of the Related Art

Electrical stimulation of various body tissues has been used for decades with documented success for repair and regeneration of bone, muscle, nervous tissue and epithelial tissue as well as increased efficacy of certain antibiotics cited in the literature.

One method to produce electrical fields is by invasively placing electrodes into the bone with the cathode (negative pole) placed into the site of bone repair and the anode (positive end) placed in the nearby soft tissue.

Non-invasive methods to produce electrical fields are by capacitive coupling (CC) and by inductive coupling (IC). IC produces electrical fields using pulsed, time varying PEMFs. This technique uses a single or double current carrying coil, which is driven by an external field generator which induces a secondary field in bone. A number of configurations can be used, both single pulse and pulse burst, the latter consisting of a series of pulses with frequencies of 1-100 pulses/sec. Depending upon amplitude, frequency, and wave-form configurations, time-varying magnetic fields of 0.1 to 20 gauss can be used, producing voltage gradients of 1-10 microvolts in the inductive search coil. Gauss is a unit used in measuring the repelling effect of a magnetic field.

Selected, weak, pulsed electromagnetic fields (PEMFs) have been successfully used in more than 250,000 patients during the past 20 years without evidence of hazardous side effects. This surgically non-invasive technique has the capacity to trigger rapid angiogenesis, to improve early bone union and to reduce bone resorption. Each of these proven bioeffects has a rational basis for improving the ultimate fate of a bone graft. Their physical principles and mechanisms of action are sufficiently well documented to place them alongside phramaceutical agents as therapeutic adjuncts to bone grafting procedures. PEMF's have a rational role in minimizing the chance for a bone graft failure.

Articles relating to bone stimulation of teeth, periodontal ligament, dental implants and intra-oral bone grafts are more limited. With orthodontics, PEMF have been shown to increase the rate of orthodontic tooth movement and bone deposition without unfavorable effects on the periodontal ligament. In vitro periodontal studies show PEMF stimulate osteoblast and fibroblast activity. In bone growth stimulation, it has been shown that an electrical charge, either direct or indirect, can promote osteogenesis and bone formation around dental implants as well as prevent ridge resorption after tooth extraction. PEMF stimulation has been found to be useful for the present invention as a result of earlier assessments and discussions of the current literature. PEMF in this context refers to the induction of voltage or current in tissues by an externally applied pulsating magnetic field.

Several medical devices are currently on the market using PEMF principles for tissue stimulation. These devices have coils imbedded into adhesive pads similar to ECG leads. The coils are attached to the power source of the device similar in appearance to an older cell phone. The “ECG” leads are attached to the skin on either side of the area to be treated. Obviously, the size and design of these devices preclude their intra-oral use.

European Patent Application No. 0,599,786, filed Apr. 7, 1993 by Enzo Lino Diodato, discloses a mouth internal device which generates a magnetic field in the oral cavity, which thereby induces an electrical field with “the result to stimulate in the tooth the accelerated growth of the dentine, with the aim of protecting the dental pulp in a series of pathological processes that can compromise its vitality.”

Japanese Patent No. JP3007172, published Jan. 4, 1991 by Hashimoto et al., discloses a device which accomplishes “a therapy free from pain and inflammation by locating teeth between two permanent magnets facing each other, feeding a pulse current to a coil for generation of an electromagnetic field in pulse form, superposing this over a steady magnetic field from the permanent magnets, and applying this resultant field to the teeth.”

These prior art devices have the disadvantage that, while being used internally, they must connect externally with a power source that powers the coil(s). This provides an awkward situation for the patient who must not only manage any discomfort of these prior art devices in their mouth, but must also deal with cables extending therefrom.

Furthermore, the secure placement of these prior art devices within the oral-cavity, such that the intended area to be treated is appropriately within the generated fields, is difficult to achieve and the patient must struggle with remaining still during the treatment period. This places an undue burden on the patient since it is possible that treatment periods can range from several hours.

### BRIEF SUMMARY OF THE INVENTION

It is, accordingly, an object of the present invention to provide a novel and improved medical device for intra-oral use that incorporates the power source within the device itself, and allows the secure placement of such a device within the oral cavity.

According to one aspect of the present invention, there is provided a medical device for therapeutic treatment within an oral cavity. The medical device comprises a means for generating an electromagnetic field for the therapeutic treatment. The means for generating an electromagnetic field includes an electrical power source to energize the means for generating the electromagnetic field. There is also a prosthetic appliance shaped to fit onto a dentition. The means for generating the electromagnetic field is integrated with the prosthetic appliance.

According to another aspect of the present invention, there is provided a method for therapeutic treatment of an area in the oral cavity. The method comprising the steps of installing a prosthetic appliance to fit onto a dentition of a patient; and providing means for generating an electromagnetic field integrated in the prosthetic appliance, the means for generating the electromagnetic field including an electrical power source, and employing the electrical power source to energize the means for generating the electromagnetic field, whereby the electromagnetic field induces a therapeutic effect in the area.

### BRIEF DESCRIPTION OF THE DRAWINGS

The invention will be more readily understood from the preferred embodiments thereof illustrated, by way of example, in the accompanying drawings, in which:

FIG. 1 shows a perspective view of a medical device for intra-oral use according to one embodiment of the present invention;

FIG. 2 shows a perspective view of an opposing pair of coils and a control unit of the medical device of FIG. 1;

FIG. 3 shows an exploded view of the control unit of FIG. 2;

FIG. 4 shows a diagrammatic view of pulsed magnetic field characteristics;

FIG. 5 shows a diagrammatic view of the medical device of FIG. 1;

FIG. 6 shows a magnetic field produced by a circular coil of the medical device of FIG. 1;

FIG. 7 shows a diagrammatic view of a magnetic field at the center of two coils of the medical device of FIG. 1;

FIG. 8 shows a graphical view of magnetic field strength of the magnetic field of the coils of FIG. 7;

FIG. 9 shows a perspective view of a medical device for intra-oral use according to another embodiment of the present invention; and

FIG. 10 shows a circuit diagram of the control unit of FIG. 2.

### DESCRIPTION OF THE PREFERRED EMBODIMENT

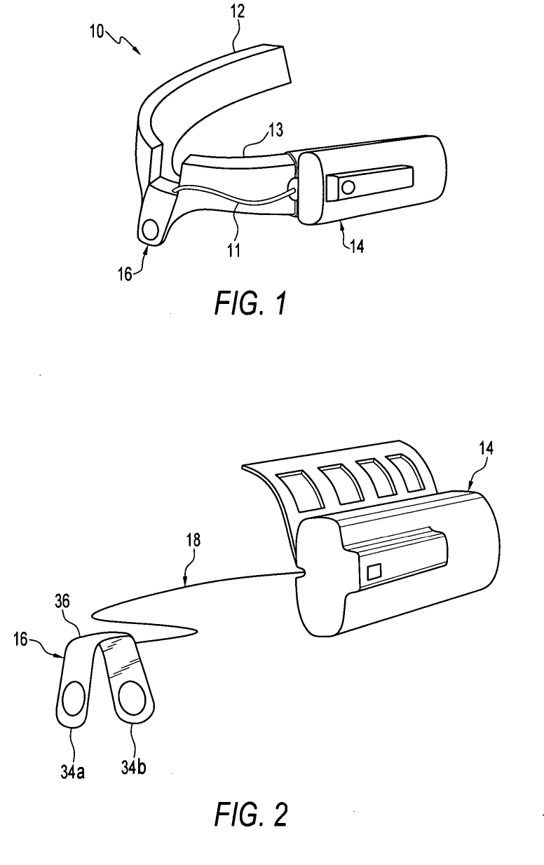

Referring to the drawings and first to FIG. 1, there is shown a medical device indicated generally by reference numeral 10, which comprises a prosthetic appliance, in this example a mouth guard 12, and a control unit 14 and a coil assembly 16. In other examples the prosthetic appliance can be a custom made prosthesis fabricated from thermoplastic or processed acrylic. The mouth guard 12 is adapted to fit onto and to conform to the dentition of a patient, and comprises a sheet of ethylvinyl acetate 11, in this example, having an arch 13.

The coil assembly 16 is in the form of a saddle bag, in this example, and is arranged to straddle the area of treatment of the patient, and therefore the position of the coil assembly 16 along the mouth guard 12 varies from patient to patient.

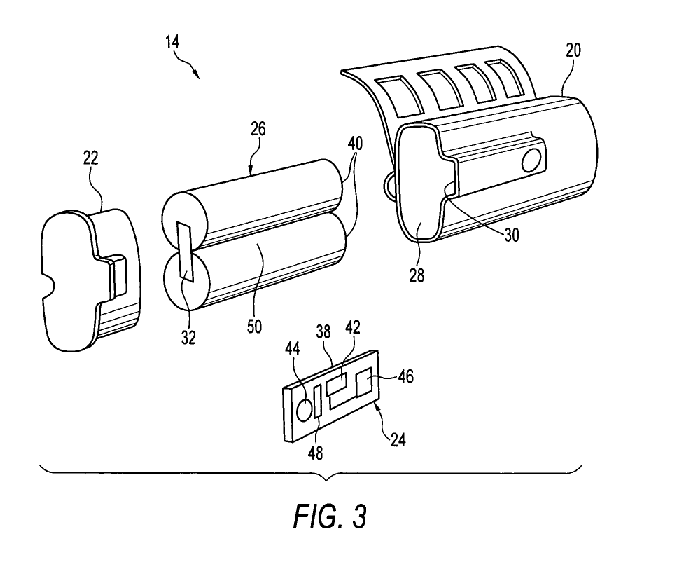

Referring now to FIGS. 2 and 3, the control unit 14 and the coil assembly 16 are shown apart from the mouth guard 12. Electrical wires 18 electrically connect the coil assembly 16 with the control unit 14. The electrical wires 18 provide a conduit for current pulses from the control unit 14 to the coil assembly 16 so that an electromagnetic field can be established, which is described in more detail below.

The coil assembly 16 includes coils 34a and 34b, which are joined together in series in this example, and which are arranged in a spaced apart and opposing manner. The coils 34a and 34b are imbedded into a water proof, plastic lamination 36 that can flex in the middle to allow the coils 34a and 34b to be placed on either side of the intended area of pulse electromagnetic field (PEMF) stimulation.

Each of the coils 34a and 34b comprises, in this example,200 windings of 39 gauge copper wire, and the inside diameter of the coils is 9mm and the outside diameter is 1 mm. The coils 34a and 34b are made by wrapping wire around a removable central core which is, in this example, 9 mm in diameter. In other examples different dimensions of the coil are possible. Once wrapped, wire turns of each of the coils 34a and 34b are bonded together with an adhesive cement.

Ends of the wire of the coils 34a and 34b are extended about 3 to 4 inches, in this example, to attach at one end to the control unit 14. The coils are then removed from the removable core and cemented onto the plastic lamination 36. The ends of the wire of the coils 34a and 34b for connection to the control unit 14 are twisted together to form the electrical wires 18.

As shown in FIG. 3, the control unit 14 comprises a housing 20, an end cap 22, a circuit board assembly 24 and a power source 26. The housing 20 has receptacles 28 and 30 for receiving the power source 26 and the circuit assembly 24, respectively.

In this embodiment of the present invention, the power source 26 includes two 1.5 Volt AAAA batteries 40, however other types of batteries can be used in other embodiments. The batteries 40 are adjacent each other with a negative pole of one of the batteries 40 being adjacent a positive pole of the other battery. A metal strip 32 connects the batteries 40 in series to form a 3 volt power source for the circuit board assembly 24 and the coils 34a and 34b. There are lead wires (not shown) which extend from the batteries 40, at an end of the batteries opposite the metal strip 32, for connection with the control unit 14. The two batteries 40 are shrink wrapped (not shown).

Referring now to both FIGS. 3 and 10, the circuit board assembly 24 includes a printed circuit board 38, an LED 42, a switch 44, a microcontroller 46 and a transistor 48. The electrical wires 18 and the lead wires from the power source 26 are connected with the printed circuit board 38 such that electrical power from the power source 26 can be selectively applied to the coils 34a and 34b.

The circuit board assembly 24 is cemented onto a surface 50 of the power source 26, and the combination is then received within the receptables 28 and 30 respectively. The control unit 14 is dipped into a waterproofing material. In other examples, the circuit board assembly 24 and the power source 26 can be affixed directly to the mouth guard 12 without first inserting them into the housing 20 and the end cap 22.

Referring again to FIG. 1, the mouth guard 12 is shaped to conform to the dentition of a patient, and the control unit 14 and the coil assembly 16 are attached with the mouth guard, according to the following procedure:
1. Take an accurate Alginate impression of the jaw of the patient, including the area of treatment. Ensure the impression is extended well into the buccal sulcus, especially in the treatment area.
2. Pour up the impression in dental stone.
3. Mold a thin sheet, e.g. twenty to sixty mil, of ethylvinyl acetate 11 onto the model using an OmniVac.
4. Fold the coil assembly 16 over the area to be treated and attach one of the coils 34a or 34b to the acetate sheet 11 using adhesive.
5. Attach the coil at the other end of the coil assembly 16 to the acetate sheet 11 onto the 10 opposite side of the arch 12, ensuring that both of the coils 34a and 34b bracket the area to be treated.
6. Attach the control unit 14 to buccal aspect of the acetate sheet 11 on the side of the arch 12. Ensure the control unit 14 lies deep in the buccal sulcus and the switch 44 faces buccally.
7. Make a cut into the acetate sheet 11 three millimeters on either side of the coil assembly 16 at right angles to the tangent of the arch 12. Extend the cut into the buccal sulcus.
8. Make a cut into the acetate sheet 11 five millimeters on either side of the control unit 14 at right angles to the tangent of the arch 12. Extend the cut into the buccal sulcus.
9. Trim the balance of the acetate sheet 11 up to the buccal sulcus.
10. Using a propane torch, gently heat the acetate sheet 11 remaining next to the coil assembly 16 on the buccal side until soft.
11. Fold the cut portion of the acetate sheet 11 back over the coil assembly 16 ensuring the fold is in the buccal sulcus.
12. Trim any excess of the acetate sheet 11 from the buccal sulcus.
13. Repeat for the lingual extension over the coil assembly 16 .
14. Repeat the same process for the extension over the control unit 14.
15. Bond the cut portions of the acetate sheet 11 together, e.g. paint a coat of adhesive over the acetate sheet 11 or heat seal.
16. Mold a second thin sheet, e.g. twenty to sixty mil, of ethylvinyl acetate over the previous sheet onto the model using an OmniVac.
17. Trim off all excess from the second sheet allowing the margins to end in the buccal and lingual sulci.
18. Using a propane torch, run the flame over edges of the two sheets to meld the two edges together.

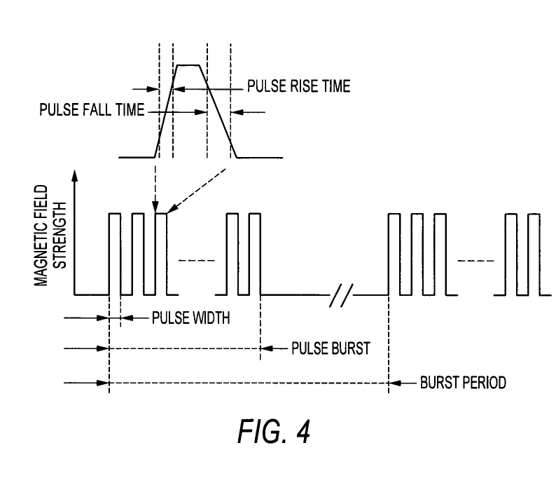

Referring now to FIG. 4, the following are exemplary PEMF characteristics for the medical device 10. However, other PEMF characteristics are possible. A waveform of the pulsed magnetic field is shown in FIG. 4. The overall duty cycle of the waveform (FIG. 4) is 5%. Treatment duration may be varied.
Pulse amplitude (magnetic field strength)=10 Gauss
Pulse width 250 s (50% duty cycle)
Number of pulses per burst=20
Burst repetition frequency=10 Hz
Treatment duration=2 hrs per day
Treatment period 8 weeks

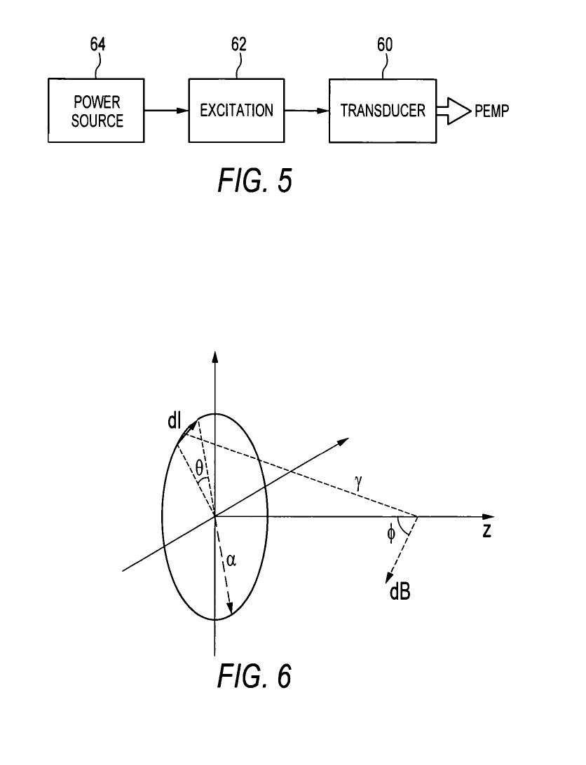

FIG. 5 is a first level architectural design of the medical device 10, illustrating a transducer 60 to produce the PEMF, an excitation circuit 62 to create the necessary current pulses to the transducer 60, and a power source 64.

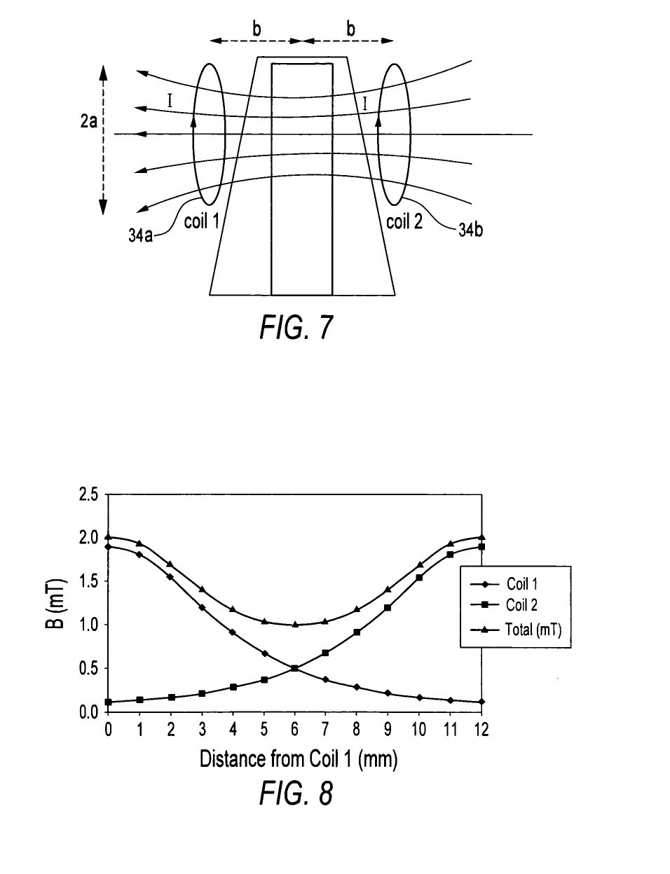

The coils 34a and 34b formed by insulated copper wire are chosen to produce the PEMF. The magnetic field of circular coils, for example, with a radius ‘a’ shown in FIGS. 6 and 7, at a distance ‘z’ along the coil axis is derived as follows. If the current flowing through a single turn coil is I, a magentic field dB is induced by the current element flowing in a small segment dl of the loop at a distance z away from the center of the coil. dB is given by:

$$dB = \frac{{μ}_{0} dl}{4 π {r}^{2}} = \frac{{μ}_{0} Iad θ}{4 π {r}^{2}}$$

The component of the magnetic field in the direction of the Z axis is:

$${dB}_{z} = - dB cos θ , where cos θ = \frac{a}{r}$$

Therefore, the magnetic field due to the entire coil in the direction of the z axis is:

$$\begin{matrix}{B}_{z} = \int {dB}_{z} \\ = {\int }_{0}^{2 π} - \frac{{μ}_{0}}{4 π} \frac{Iad θ}{{r}^{2}} \frac{a}{r} \\ = - \frac{{μ}_{0} {Ia}^{2}}{4 π {r}^{3}} {\int }_{0}^{2 π} ⅆ θ \\ = - \frac{{μ}_{0} {Ia}^{2}}{4 π {r}^{3}} 2 π \\ = - \frac{{μ}_{0} {Ia}^{2}}{2 {r}^{3}}\end{matrix}$$

For an N-turn coil, the magnetic field becomes:

$${B}_{z} = - \frac{{μ}_{0} {NIa}^{2}}{2 {r}^{3}} , but r = \sqrt{{a}^{2} + {z}^{2}} , therefore$$

$${B}_{z} = \frac{{μ}_{0} {NIa}^{2}}{2 {( {a}^{2} + {z}^{2} )}^{\frac{3}{2}}}$$

As the transducer 60 must be totally imbedded into the mouth guard 12, very thin insulated copper wire is needed to form the coils 34a and 34b to produce the magnetic field. FIG. 7 shows the coils 34a and 34b placed at b mm away from the center on each side of the dental implant. Note that a typical titanium dental implant is 4 mm in diameter and 8 to 15 mm long. If the current in both of the coils 34a and 34b is equal (I) and flowing in the same direction, from equation (1), the magnetic field B at the center is equal to:

$$B = {B}_{1} + {B}_{2} = 2 {B}_{z} = 2 \frac{{μ}_{0} {NIa}^{2}}{2 {( {a}^{2} + {b}^{2} )}^{\frac{3}{2}}} = \frac{{μ}_{0} {NIa}^{2}}{{( {a}^{2} + {b}^{2} )}^{\frac{3}{2}}} \qquad ( 2 )$$

where B₁ and B_{2 are the magnetic fileds due to coil 34}a and coil 34b in FIG. 7 respectively

When each of the coils 34a and 34b has 200 turns (N=200) with a diameter of 10 mm (a=5 mm), and the coils are separated by 12 mm (b=6 mm). For non-ferromagnetic material, the permeability is approximately equal to that in free space, i.e., μ₀=4π×10⁻⁷ T.m/A. To produce a magnetic field of 10 Gauss (B=1 mT), using equation (2) above:

$$B = \frac{{μ}_{0} {NIa}^{2}}{{( {a}^{2} + {b}^{2} )}^{\frac{3}{2}}} ⇒ I = \frac{{( {a}^{2} + {b}^{2} )}^{\frac{3}{2}}}{{μ}_{0} {Na}^{2}} B$$

and substituting the chosen values into the equation, the excitation current I is 76 mA.

Table 1 shows the calculated magnetic field strength along the central axis of the setup in FIG. 7 when I=76 mA. The magnetic field due to coil 34a alone, coil 34b alone and both coils at a distance of b₁ from the left coil are tabulated in the table. FIG. 8 shows the plot of these magnetic fields at different locations along the center axis of the coils.

TABLE 1

| b1(mm) | Coil 34a | Coil 34b | Total (mT) |
|---|---|---|---|
| 0 | 1.91 | 0.11 | 2.01 |
| 1 | 1.8 | 0.14 | 1.93 |
| 2 | 1.53 | 0.17 | 1.7 |
| 3 | 1.2 | 0.22 | 1.42 |
| 4 | 0.91 | 0.28 | 1.19 |
| 5 | 0.67 | 0.37 | 1.05 |
| 6 | 0.5 | 0.5 | 1 |
| 7 | 0.37 | 0.67 | 1.05 |
| 8 | 0.28 | 0.91 | 1.19 |
| 9 | 0.22 | 1.2 | 1.42 |
| 10 | 0.17 | 1.53 | 1.7 |
| 11 | 0.14 | 1.8 | 1.93 |
| 12 | 0.11 | 1.91 | 2.01 |

As the waveform duty cycle is 5%, the average current consumption I_{avg} is therefore equal to 76 mA×5%=3.8 mA.

Since the device is desired to be used for 2 hrs/day for an 8 week period, the minimum capacity of the power source supplying power to the device is therefore equal to (2 hr/day×7 day/wk×8 wk)×3.8 mA=426 mAhr.

This is within the energy capacity of a lithium AAAA cylindrical battery (which has a typical capacity of 1250 mAhr and 1.5V output).

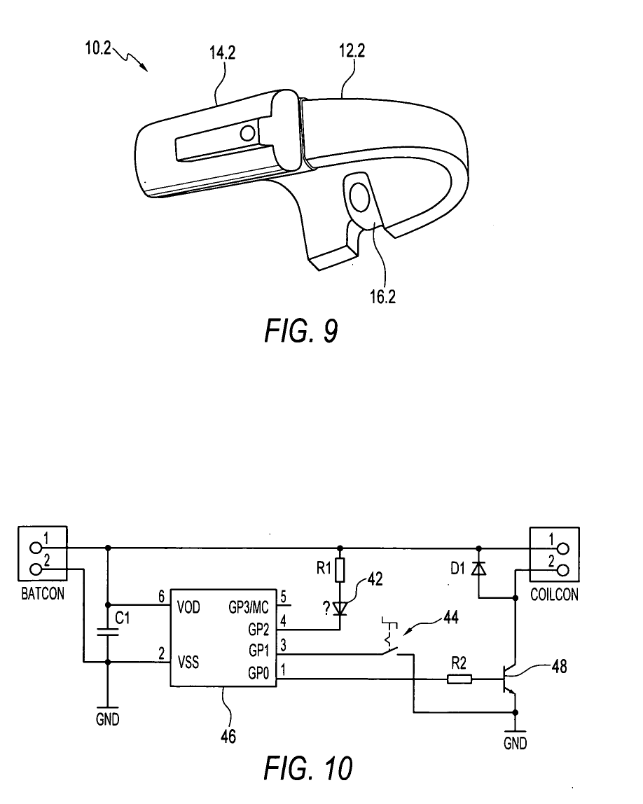

Referring now to FIG. 9, wherein like parts to the previous embodiment have like reference numerals with an additional suffix “0.2“, there is shown a medical device 10.2 having a control unit 14.2 and a coil assembly 16.2 at different locations along a mouth guard 12.2, in contrast to the medical device 10, for treatment in another area of the dentition.

As will be apparent to those skilled in the art, various modifications may be made in the present invention within the scope of the appended claims.

I claim:

1. A medical device for therapeutic treatment within an oral cavity, comprising:
means for generating an electromagnetic field for the therapeutic treatment, the means for generating an electromagnetic field including an electrical power source to energize the means for generating the electromagnetic field; and
a prosthetic appliance shaped to fit onto a dentition, the means for generating the electromagnetic field being integrated with the prosthetic appliance.

2. The medical device as claimed in claim 1, wherein the means for generating the electromagnetic field includes:
a first coil; and
means for controlling the medical device, said means for controlling selectively electrically connecting the electrical power source with the first coil, and the electromagnetic field being generated when the electrical power source is connected with the first coil.

3. The medical device as claimed in claim 2, wherein the means for generating the electromagnetic field further comprises a second coil, the first coil opposing the second coil in a spaced apart relationship, and the means for controlling selectively electrically connecting the electrical power source with the second coil.

4. The medical device as claimed in claim 3, wherein the prosthetic appliance is adapted to extend from a buccal side of the dentition towards a lingual side of the dentition, thereby enveloping the dentition, the first coil being integrated in the prosthetic appliance on the buccal side of the dentition and the second coil being integrated in the prosthetic appliance on the lingual side of the dentition.

5. The medical device as claimed in claim 1, wherein the means for generating the electromagnetic field includes means for automatically shutting off the electromagnetic field after a predetermined period of time.

6. The medical device as claimed in claim 2, wherein the means for controlling includes means for selectively disconnecting the power source from the first coil after a predetermined period of time.

7. The medical device as claimed in claim 1, wherein the prosthetic appliance is a mouth guard.

8. A method for therapeutic treatment of an area in the oral cavity, the method comprising the steps of:
installing a prosthetic appliance to fit onto a dentition of a patient; and
providing means for generating an electromagnetic field integrated in the prosthetic appliance, the means for generating the electromagnetic field including an electrical power source; and
employing the electrical power source to energize the means for generating the electromagnetic field, whereby the electromagnetic field induces a therapeutic effect in the area.

9. The method for therapeutic treatment as claimed in claim 8, wherein the means for generating the electromagnetic field includes selectively electrically connecting the electrical power source with a first coil, the electromagnetic field being generated when the electrical power source is connected with the first coil.

10. The method for therapeutic treatment as claimed in claim 9, which includes installing the prosthetic appliance so that the prosthetic appliance extends from a buccal side of the dentition towards a lingual side of the dentition, the first coil being integrated in the prosthetic appliance on the buccal side of the dentition and a second coil being integrated in the prosthetic appliance on the lingual side of the dentition.

11. The method for therapeutic treatment as claimed in claim 10, which includes automatically shutting off the electromagnetic field after a predetermined period of time.
